SEQUENCE LISTING (10 sequences)

SEQ ID NO: 1 - DNA, 2220 bp, Artificial Sequence
  Description of Artificial Sequence: Synthetic MVA 65A/G Env polynucleotide
atgagagtgatggggatacagaagaattatccactcttatggagagggggtatgacaatattttggttaatgatgatttgtaatgctgaaaagttgtgggtcacagtctactatggggtacctgtgtggagagacgcagagaccaccctattctgtgcatcagatgctaaagcatatgacaaagaagcacacaatgtctgggctacgcatgcctgcgtacccacagaccctgacccacaagaattacctttggtaaatgtaacagaagagtttaacatgtggaaaaataatatggtagaacagatgcatgaagatataattagtctatgggaccaaagcttaaagccatgtgtacagctaacccctctctgcgttactttagggtgtgctgacgctcaaaacgtcaccgacaccaacaccaccatatctaatgaaatgcaaggggaaataaaaaactgctctttcaatatgaccacagaattaagagataagaagcagaaagtgtatgcacttttctatagacctgatgtaatagaaattaataaaactaagattaacaatagtaatagtagtcagtatatgttaataaattgtaatacctcaaccattacacagacttgtccaaaggtatcctttgagccaattcccatacattattgtgccccagctggttttgcaattctaaagtgtaatgatacggagttcagtggaaaagggacatgcaagagtgtcagcacagtacaatgcacacatggaatcaagccagtagtatcaactcaactgctgttaaatggcagtctagcagaaggaaagatagcgattagatctgagaatatctcaaacaatgccaaaactataatagtacaattgactgagcctgtagaaattaattgtatcagacctggcaacaatacaagaaaaagtgtacgcataggaccaggacaaacattctatgcaacaggtgacataataggagatataagacaagcacactgtaatgttagtaaaatagcatgggaagaaactttacaaaaggtagctgcacaattaaggaagcactttcagaatgccacaataaaatttactaaacactcaggaggggatttagaaattacaacacatagttttaattgtggaggagaattcttctattgcaatacaacaaagctgtttaatagcacttggaataatgataactcaaacctcacagaggaaaagagaaaggaaaacataactctccactgcagaataaagcaaattgtaaatatgtggcagagagtaggacaagcaatatatgcccctcccatcccaggaaacataacttgtggatcaaacattactgggctactattaacaagagatggagggaataatggtacaaatgatactgagaccttcaggcctggaggaggagatatgagggacaattggagaagtgaattatataaatataaagtagtaaaaattgaaccactaggtgtagcaccaacccctgcaaaaagaagagtggtggaaagagaaaaaagagcagttggaatgggagctttgatctttgagttcttaggagcagcaggaagcactatgggcgcggcgtcaatggcgctgacggtacaggccagacaattattgtctggtatagtgcaacagcagagcaatctgctgaaggctatagaggctcaacaacatctgttgagactcacggtctggggcattaaacagctccaggcaagagtcctggctctggaaagatacctaaaggatcaacagctcctaggaatttggggctgctctggaaaactcatttgcaccactgctgtaccttggaactctagctggagtaataaaagttataatgacatatgggataacatgacctggctgcaatgggataaagaaattaacaattacacatacataatatataatctacttgaaaaatcgcagaaccagcaggaaattaatgaacaagacttattggcattagacaagtgggcaagtctgtggaattggtttgacataacaagctggctatggtatataagattaggtataatgatagtaggaggcgtaataggcttaagaataatttttgctgtgcttactatagtgaatagagttaggcagggatactcacctttgtcattccagacccttgcccaccaccagagggaacccgacaggcccgaaagaatcgaagaaggaggtggcgagcaagactaa

SEQ ID NO: 2 - DNA, 3473 bp, Artificial Sequence
  Description of Artificial Sequence: Synthetic MVA 65A/G GagPol polynucleotide
atgggtgcgagagcgtcagtgttaacggggggaaaattagattcatgggagaaaattaggttaaggccagggggaaagaaaagatatagactaaaacacctagtatgggcaagcagggagctggagagattcgcacttaaccctggcctattagaaacagcagaaggatgtcaacaactaatgggacagttacaaccagctctcaggacaggatcagaagagtttaaatcattatataatatagtagcaaccctttggtgcgtacatcaaagaatagacataaaagacacccaggaggccttagataaagtagaggaaaaacaaaataagagcaagcaaaaggcacagcaggcagcagctgcaacagccgccacaggaagcagcagccaaaattaccctatagtgcaaaatgcacaagggcaaatggtacatcagtccatgtcacctaggactttaaatgcatgggtgaaggtaatagaagaaaaggcttttagcccagaggtaatacccatgttttcagcattatcagagggagccaccccacaagatttaaatatgatgctaaacatagtggggggacaccaggcagcaatgcagatgttaaaagataccatcaatgatgaagctgcagaatgggacagagtacatccagtacatgcagggcctattccaccaggccaaatgagggaaccaaggggaagtgacatagcaggaactactagtacccttcaagaacaaataggatggatgacaagtaatccacctatcccagtgggagaaatctataaaagatggatagtcctgggattaaataaaatagtaagaatgtatagccctaccagcattttggacataagacaagggccaaaagaaccctttagagattatgtagacaggttctttaaaactttgagagctgaacaagctacgcaggaggtaaaaaactggatgacagaaaccttgttggtccaaaatgcgaatccagactgcaagtccattttaagagcattaggaccaggggctacattagaagaaatgatgacatcatgtcagggagtgggaggacctggccataaagcaagggttttggctgaggcaatgagtcaagtacaacagaccaatgtaatgatgcagagaggcaattttagaggccagagaataataaagtgtttcaactgtggcaaagaaggacacctagccagaaattgcaaggctcctagaaagagaggctgttggaaatgtggaaaggaaggacaccaaatgaaagactgtactgaaagacaggctaattttttagggaaaatttggccttcccacaaggggaggccaggaaattttcctcagagcagaccagaaccaacagccccgccagcagagagctttggagtgggggaagagataccctcctctccgaagcaggagccgagggacaagggactatatcctcccttaacttccctcaaatcactctttggcaacgaccagtagtcacagtaagaatagggggacagccaatagaagccctattagacacaggagcagatgatacagtattagaagaaataagtttaccaggaaaatggaaaccaaaaatgatagggggaattggaggttttatcaaagtaagacagtatgatcagatatctatagaaatttgtggaaaaagggccataggtacagtattagtaggacctacacctgtcaacataattggacgaaatatgttgactcagattggttgtactttaaattttccaattagtcctattgaaactgtgccagtaaaattaaagccaggaatggatggcccaaaggttaaacaatggccattgacagaagaaaaaataaaagcattaaaagaaatttgtgcagagatggaaaaggaaggaaaaatttcaaaaattgggcctgaaaacccatacaatactccaatatttgccataaagaaaaaagatagtactaaatggagaaaattagtagatttcagagaactcaataagagaactcaagacttctgggaggtccaattaggaatacctcatcctgcgggattaaaaaagaaaaaatcagtaacagtactagatgtgggggatgcatatttttcagttcccttagatgaagactttagaaaatatactgcattcaccatacctagtttaaataatgagacaccagggattagatatcagtacaatgtactcccacagggatggaaaggatcaccagcaatatttcaggcaagcatgacaaaaatcttagagccctttagagcaaaaaatccagagatagtgatctaccaatatatgaacgatttatatgtaggatctgacttagaaatagggcagcatagagcaaaaatagaggagttgagagaacatctattgaaatggggatttaccacaccagacaaaaaacatcagaaagaacctccatttctttggatgggatatgaactccatcctgacaaatggacagtccagcctatacagctgccagaaaaagacagctggactgtcaatgatatacaaaaattagtgggaaaactaaataccgcaagtcagatttatgcaggaattaaagtaaagcaattgtgtagactcctcaggggagccaaagcgctaacagatgtagtaacactgactgaggaagcagaattagaattggcagagaacagggaaattctaaaagaacctgtacatggagtatattatgacccaacaaaagacttagtggcagaaatacagaaacaagggcaagatcaatggacatatcaaatttatcaagagccatttaaaaatctaaagacaggaaaatatgcaaaaaagaggtcggcccacactaatgatgtaaaacaattaacagaggtagtgcagaaaatagccatagaaagcatagtaatatggggaaagacccctaaatttagactacccatacaaagagaaacatgggaagcatggtggatggagtattggcaggctacctggattcctgaatgggagtttgtcaatacccctcctctagtaaaattatggtaccagttagagaaggaccccataatgggagcagaaactttctatgtagatggggcagctaatagggagactaagctaggaaaagcagggtatgtcactgacagaggaagacaaaaggttgtttccctaattgagacaacaaatcaaaagactcagttacatgcaattcatctagccttgcaggattcaggatcagaagtaaatatagtaacagactcacagtatgcattaggaatcattcaggcacaaccagacaggagtgaatcagagttagtcaatcaaataatagagaaactaatagaaaaggacaaagtctacctgtcatgggtaccagcacacaaagggattggaggaaatgaacaagtagataaattagtcagtagtggaatcagaaaggtactatttttagatggaatagataaagcccaagatgaacattag

SEQ ID NO: 3 - DNA, 2217 bp, Artificial Sequence
  Description of Artificial Sequence: Synthetic MVA 62B ADA env polynucleotide
atgaaagtgaaggggatcaggaagaattatcagcacttgtggaaatggggcatcatgctccttgggatgttgatgatctgtagtgctgtagaaaatttgtgggtcacagtttattatggggtacctgtgtggaaagaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttctatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagacaagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagaaataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaattcttctactgtaattcaacacaactgtttaatagtacttggaattttaatggtacttggaatttaacacaatcgaatggtactgaaggaaatgacactatcacactcccatgtagaataaaacaaattataaatatgtggcaggaagtaggaaaagcaatgtatgcccctcccatcagaggacaaattagatgctcatcaaatattacagggctaatattaacaagagatggtggaactaacagtagtgggtccgagatcttcagacctgggggaggagatatgagggacaattggagaagtgaattatataaatataaagtagtaaaaattgaaccattaggagtagcacccaccaaggcaaaaagaagagtggtgcagagagaaaaaagagcagtgggaacgataggagctatgttccttgggttcttgggagcagcaggaagcactatgggcgcagcgtcaataacgctgacggtacaggccagactattattgtctggtatagtgcaacagcagaacaatttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaaactctggatatgatttgggataacatgacctggatggagtgggaaagagaaatcgaaaattacacaggcttaatatacaccttaattgaggaatcgcagaaccaacaagaaaagaatgaacaagacttattagcattagataagtgggcaagtttgtggaattggtttgacatatcaaattggctgtggtatgtaaaaatcttcataatgatagtaggaggcttgataggtttaagaatagtttttactgtactttctatagtaaatagagttaggcagggatactcaccattgtcatttcagacccacctcccagccccgaggggacccgacaggcccgaaggaatcgaagaagaaggtggagacagagactaa

SEQ ID NO: 4 - DNA, 3479 bp, Artificial Sequence
  Description of Artificial Sequence: Synthetic MVA 62B GagPol
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgagaatccatacaatactccagtatttgccataaagaaaaaagacagtactaaatggaggaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttaaaaaacaaaatccagacatagttatctatcaatacatgaacgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaataccgcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaacaaaggaagacaaaaggttgtccccctaactaacacaacaaatcagaaaactcagttacaagcaatttatctagctttgcaggattcaggattagaagtaaacatagtaacagactcacaatatgcattaggaatcattcaagcacaaccagataaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaaatactatttttagatggaatagataaggcccaagatgaacattag

SEQ ID NO: 5 - DNA, 2244 bp, Artificial Sequence
  Description of Artificial Sequence: Synthetic MVA/HIV 71C Env polynucleotide
atgagagtgaaggggatactgaggaattatcgacaatggtggatatggggcatcttaggcttttggatgttaatgatttgtaatggaaacttgtgggtcacagtctattatggggtacctgtgtggaaagaagcaaaaactactctattctgtgcatcaaatgctaaagcatatgagaaagaagtacataatgtctgggctacacatgcctgtgtacccacagaccccaacccacaagaaatggttttggaaaacgtaacagaaaattttaacatgtggaaaaatgacatggtgaatcagatgcatgaggatgtaatcagcttatgggatcaaagcctaaagccatgtgtaaagttgaccccactctgtgtcactttagaatgtagaaaggttaatgctacccataatgctaccaataatggggatgctacccataatgttaccaataatgggcaagaaatacaaaattgctctttcaatgcaaccacagaaataagagataggaagcagagagtgtatgcacttttctatagacttgatatagtaccacttgataagaacaactctagtaagaacaactctagtgagtattatagattaataaattgtaatacctcagccataacacaagcatgtccaaaggtcagttttgatccaattcctatacactattgtgctccagctggttatgcgattctaaagtgtaacaataagacattcaatgggacaggaccatgcaataatgtcagcacagtacaatgtacacatggaattaagccagtggtatcaactcagctattgttaaacggtagcctagcagaaggagagataataattagatctgaaaatctgacagacaatgtcaaaacaataatagtacatcttgatcaatctgtagaaattgtgtgtacaagacccaacaataatacaagaaaaagtataaggatagggccaggacaaacattctatgcaacaggaggcataatagggaacatacgacaagcacattgtaacattagtgaagacaaatggaatgaaactttacaaagggtgggtaaaaaattagtagaacacttccctaataagacaataaaatttgcaccatcctcaggaggggacctagaaattacaacacatagctttaattgtagaggagaattcttctattgcagcacatcaagactgtttaatagtacatacatgcctaatgatacaaaaagtaagtcaaacaaaaccatcacaatcccatgcagcataaaacaaattgtaaacatgtggcaggaggtaggacgagcaatgtatgcccctcccattgaaggaaacataacctgtagatcaaatatcacaggaatactattggtacgtgatggaggagtagattcagaagatccagaaaataataagacagagacattccgacctggaggaggagatatgaggaacaattggagaagtgaattatataaatataaagcggcagaaattaagccattgggagtagcacccactccagcaaaaaggagagtggtggagagagaaaaaagagcagtaggattaggagctgtgttccttggattcttgggagcagcaggaagcactatgggcgcagcgtcaataacgctgacggtacaggccagacaattgttgtctggtatagtgcaacagcaaagcaatttgctgagggctatcgaggcgcaacagcatctgttgcaactcacggtctggggcattaagcagctccagacaagagtcctggctatcgaaagatacctaaaggatcaacagctcctagggctttggggctgctctggaaaactcatctgcaccactaatgtaccttggaactccagttggagtaacaaatctcaaacagatatttgggaaaacatgacctggatgcagtgggataaagaagttagtaattacacagacacaatatacaggttgcttgaagactcgcaaacccagcaggaaagaaatgaaaaggatttattagcattggacaattggaaaaatctgtggaattggtttagtataacaaactggctgtggtatataaaaatattcataatgatagtaggaggcttgataggcttaagaataatttttgctgtgctttctatagtgaatagagttaggcagggatactcacctttgtcgtttcagacccttaccccaaacccaaggggacccgacaggctcggaagaatcgaagaagaaggtggagggcaagacagagactaa

SEQ ID NO: 6 - DNA, 3419 bp, Artificial Sequence
  Description of Artificial Sequence: Synthetic MVA/HIV 71C GagPol polynucleotide
atgggtgcgagagcgtcaatattaagagggggaaaattagataaatgggaaaagattaggttaaggccagggggaaagaaacactatatgctaaaacacctagtatgggcaagcagggagctggaaagatttgcacttaaccctggccttttagagacatcagaaggctgtaaacaaataataaaacagctacaaccagctcttcagacaggaacagaggaacttaggtcattattcaatgcagtagcaactctctattgtgtacatgcagacatagaggtacgagacaccaaagaagcattagacaagatagaggaagaacaaaacaaaagtcagcaaaaaacgcagcaggcaaaagaggctgacaaaaaggtcgtcagtcaaaattatcctatagtgcagaatcttcaagggcaaatggtacaccaggcactatcacctagaactttgaatgcatgggtaaaagtaatagaagaaaaagcctttagcccggaggtaatacccatgttcacagcattatcagaaggagccaccccacaagatttaaacaccatgttaaataccgtggggggacatcaagcagccatgcaaatgttaaaagataccatcaatgaggaggctgcagaatgggatagattacatccagtacatgcagggcctgttgcaccaggccaaatgagagaaccaaggggaagtgacatagcaggaactactagtaaccttcaggaacaaatagcatggatgacaagtaacccacctattccagtgggagatatctataaaagatggataattctggggttaaataaaatagtaagaatgtatagccctgtcagcattttagacataagacaagggccaaaggaaccctttagagattatgtagaccggttctttaaaactttaagagctgaacaagcttcacaagatgtaaaaaattggatggcagacaccttgttggtccaaaatgcgaacccagattgtaagaccattttaagagcattaggaccaggagctacattagaagaaatgatgacagcatgtcaaggagtgggaggacctagccacaaagcaagagtgttggctgaggcaatgagccaaacaggcagtaccataatgatgcagagaagcaattttaaaggctctaaaagaactgttaaatgcttcaactgtggcaaggaagggcacatagctagaaattgcagggcccctaggaaaaaaggctgttggaaatgtggaaaggaaggacaccaaatgaaagactgtgctgagaggcaggctaattttttagggaaaatttggccttcccacaaggggaggccagggaatttccttcagaacaggccagagccaacagccccaccagcagagagcttcaggttcgaggagacaacccctgctccgaagcaggagctgaaagacagggaacccttaacctccctcaaatcactctttggcagcgaccccttgtctcaataaaaatagggggccagataaaggaggctctcttagacacaggagcagatgatacagtattagaagaaatgaatttgccaggaaaatggaaaccaaaaatgataggaggaattggaggttttatcaaagtaagacagtatgatcaaatacttatagaaatttgtggaaaaaaggctataggtacagtattagtaggacccacacctgtcaacataattggaagaaatatgctgactcagattggatgcacgctaaattttccaattagtcccattgaaactgtaccagtaaaattaaagccaggaatggatggcccaaaggttaaacaatggccattgacagaggagaaaataaaagcattaacagcaatttgtgatgaaatggagaaggaaggaaaaattacaaaaattgggcctgaaaatccatataacactccaatattcgccataaaaaagaaggacagtactaagtggagaaaattagtagatttcagagaacttaataaaagaactcaagacttctgggaagttcaattaggaataccacacccagcagggttaaaaaagaaaaaatcagtgacagtactagatgtgggggatgcatatttttcagttcctttagatgaaagctttaggaggtatactgcattcaccatacctagtagaaacaatgaaacaccagggattagatatcaatataatgtgcttccacaaggatggaaaggatcaccagcaatattccagagtagcatgacaaaaatcttagagccctttagagcacaaaatccagaaatagtcatctatcaatatatgaatgacttgtatgtaggatctgacttagaaatagggcaacatagagcaaagatagaggaattaagagaacatctattaaggtggggatttaccacaccagacaagaaacatcagaaagaacccccatttctttggatggggtatgaactccatcctgacaaatggacagtacagcctatacagctgccagaaaaggagagctggactgtcaatgatatacagaagttagtgggaaaattaaacacggcaagccagatttacccagggattaaagtaagacaactttgtagactccttagaggggccaaagcactaacagacatagtaccactaactgaagaagcagaattagaattggcagagaacagggaaattctaaaagaaccagtacatggagtatattatgacccttcaaaagacttgatagctgaaatacagaaacagggacatgaccaatggacatatcaaatttaccaagaaccattcaaaaatctgaaaacagggaagtatgcaaaaatgaggactgcccacactaatgatgtaaaacggttaacagaggcagtgcaaaaaatagccttagaaagcatagtaatatggggaaagattcctaaacttaggttacccatccaaaaagaaacatgggagacatggtggactgactattggcaagccacctggattcctgagtgggaatttgttaatactcctcccctagtaaaattatggtaccagctagagaaggaacccataataggagtagaaactttctatgtagatggagcagctaatagggaaaccaaaataggaaaagcagggtatgttactgacagaggaaggcagaaaattgtttctctaactgaaacaacaaatcagaagactcaattacaagcaatttatctagctttgcaagattcaggatcagaagtaaacatagtaacagactcacagtatgcattaggaattattcaagcacaaccagataagagtgaatcagggttagtcaaccaaataatagaacaattaataaaaaaggaaagggtctacctgtcatgggtaccagcacataaaggtattggaggaaatgaacaagtagacaaattagtaagtagtggaatcaggagagtgctatag

SEQ ID NO: 7-9 - skipped (3 entries)

SEQ ID NO: 10 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

(19) United States
(12) Patent Application Publication
(10) Pub. No.: US 2013/0280215 A1
(43) Pub. Date: Oct. 24, 2013

(54) MVA VECTORS EXPRESSING POLYPEPTIDES AND HAVING HIGH LEVEL PRODUCTION IN CERTAIN CELL LINES

(71) Applicant: GeoVax, Inc., Smyrna, GA (US)

(72) Inventor: Harriet L. Robinson, Atlanta, GA (US)

(73) Assignee: GeoVax, Inc., Smyrna, GA (US)

(21) Appl. No.: 13/655,294

(22) Filed: Oct. 18, 2012

Related U.S. Application Data
(60) Provisional application No. 61/548,441, filed on Oct. 18, 2011.

(51) Int. Cl. C12N 15/86

(52) U.S. Cl. CPC C12N 15/86
USPC 424/93.2; 435/320.1

(57) ABSTRACT

The present invention provides viral vectors, such as recombinant MVA vectors, that are capable of expressing one or more polypeptides, such as, e.g., HIV proteins or GM-CSF, in the cells of a human patient at relatively high levels and can also be produced in significant quantities in cultured cells. Also provided are methods for producing the viral vectors and pharmaceutical compositions containing them.

### RELATED APPLICATIONS

This application claims the benefit of U.S. Provisional Application Ser. No. 61/548,441 filed Oct. 18, 2011, the content of which is incorporated herein by reference in its entirety.

### TECHNICAL FIELD

This invention relates to MVA vectors and vaccine inserts that are able to be produced at high levels in certain cell lines.

### BACKGROUND

Vaccines have had profound and long lasting effects on world health. Smallpox has been eradicated, polio is near elimination, and diseases such as diphtheria, measles, mumps, pertussis, and tetanus are contained. A Vaccines under development include DNA vaccines and various live vectored vaccines (e.g. Adenovirus type 5 vectors, poxvirus vectors). Live viral vectors can be used either alone or as the boost componenet for a DNA prime or a prime by another live viral vector Modified vaccinia Ankara (MVA) has been particularly effective as a boost for DNA primes in mouse models, non human primates, and humans (Schneider et al., Nat. Med. 4:397-402, 1998, Lai et al, J. Inf. Dis. 204:164-173, 2011, Goepfert et al., J. Inf. Dis. 203:610-619, 2011). MVA is a highly attenuated strain of vaccinia virus that was developed toward the end of the campaign for the eradication of smallpox, and it has been safety tested in more than 120,000 people (Mahnel et al., Berl. Munch Tierarztl Wochenschr 107:253-256, 1994; Mayr et al., Zentralbl. Bakteriol. 167:375-390, 1978). During over 500 passages in chicken cells, MVA lost about 10% of its genome and the ability to replicate efficiently in primate cells. Despite its limited replication, MVA has proved to be a highly effective expression vector (Sutter et al., Proc. Natl. Acad. Sci. U.S.A. 89:10847-10851, 1992), raising protective immune responses in primates for parainfluenza virus (Durbin et al. J. Infect. Dis. 179:1345-1351, 1999), measles (Stittelaar et al. J. Virol. 74:4236-4243, 2000), and immunodeficiency viruses (Barouch et al., J. Virol. 75:5151-5158, 2001; Ourmanov et al., J. Virol. 74:2740-2751, 2000; Amara et al., J. Virol. 76:7625-7631, 2002). The relatively high immunogenicity of MVA has been attributed in part to the loss of several viral anti-immune defense genes (Blanchard et al., J. Gen. Virol. 79:1159-1167, 1998). Vaccinia viruses have been used to engineer viral vectors for recombinant gene expression and as recombinant live vaccines (Mackett et al., Proc. Nati. Acad. Sci. U.S.A. 79:7415-7419; Smith et al., Biotech. Genet. Engin. Rev. 2:383-407, 1984). DNA sequences, which may encode any of the HIV polypeptides described herein, can be introduced into the genomes of vaccinia viruses. If the gene is integrated at a site in the viral DNA that is non-essential for the life cycle of the virus, it is possible for the newly produced recombinant vaccinia virus to be infectious (i.e., able to infect foreign cells) and to express the integrated DNA sequences. The prevalence of HIV infection has made vaccine development for this recently emergent agent a high priority for world health. The development of safe and effective vaccines against existing and emerging pathogens is a major focus of medical research. Considerable effort has been directed to making a vaccine that will protect against human immunodeficiency virus-1 (HIV). Certain MVA vectors expressing HIV polypeptides have been suggested as useful for eliciting an immune response to HIV. It is desirable to be able to produce useful quantities of MVA in cell lines.

### SUMMARY

The present invention provides viral vectors (e.g., recombinant MVA vectors) that are capable of expressing one or more (e.g., two, three, four, five, six, seven, eight, nine, or ten) polypeptides (e.g., HIV proteins or portions thereof and other human genes that modulate immune responses such as GM-CSF (granulocyte-macrophage colony stimulating factor; GenBank NP_{—}000749) in the cells of a human patient at relatively high levels and can also be produced in significant quantities in cultured cells, e.g., avian cells. The vectors include miRNA target sequences that suppress translation (expression) of the polypeptides in a selected cell type, e.g., a cell used to produce MVA for preparation of an immunogenic composition, but do not significantly reduce expression in one or more other selected cell types (e.g., mammalian cells or human cells). Thus, polypeptide expression is suppressed during production of bulk quantities of MVA, thereby permitting production of large quantities of MVA, and polypeptide expression is not suppressed in a patient to which the MVA is administered.

miRNA Target Sequences

The viral vectors include one or more miRNA target sequences in the 3′ untranslated region of the transcript(s) encoding the polypeptides (or the transcript or transcripts encoding the polypeptides and other human genes that modulate immune responses). The miRNA target sequences are selected to repress expression of the polypeptides (or polypeptides and GM-CSF) at the translational levels in the cell line used for production of the MVA while not doing so (or doing so to a lesser extent) in certain human cells, e.g., certain cells of a human patient to which the MVA is administered. Thus, the miRNA target sequences can decrease expression (e.g., by 20%, 40%, 60%, 80%, 90% or more) of the one or more HIV polypeptides (or polypeptides and immune modulator) compared to an otherwise identical MVA vector lacking the miRNA target sequence. In some cases the 3′ untranslated region includes multiple copies of one or more miRNA target sequences. Thus, the region can include at least 1, 2, 3, 4, 5, 6, 8, 10 or more copies of a first miRNA target sequence that is functional in a cell line used to produce the MVA in quantities for producing a pharmaceutical formulation. The 3′ untranslated region can also include at least 1, 2, 3, 4, 5, 6, 8, 10 or more copies of a second (different) miRNA target sequence that is functional in a cell line used to produce the MVA in quantities for producing a pharmaceutical formulation.

While it can be advantageous to include multiple miRNA target sequences in order to more full repress expression, the presence of multiple target sequences can cause undesirable recombination. Thus, it can be desirable to have a combination of 2 or more (2, 3, 4 or 5) different miRNA target sequences in order to reduce risk of recombination events while still having multiple miRNA target sequences.

In some cases the miRNA target sequence does not decrease expression of the polypeptides (or polypeptides and GM-CSF) in a cell line used for recombination of the MVA virus (e.g., chicken embryo fibroblasts could include DF1 cells, an immortalized chicken cell line 5 or BHK-21 cells, baby hamster kidney cells lines or other cell line that are sufficiently permissive for MVA growth for production of recombinants). Two broad categories of miRNA targets sequences have been identified: 5′ dominant sites and 3′ compensatory sites. Use of entire targets has been effective in suppressing transgene expression (Brown et a. 2006 Nat Med 12:585-591). One can identify active miRNA sequences for any given cell type. Vendor such as LC Sciences (Houston, Tex.) can profile mRNA isolated from a given cell type and identify miRNA target sequences likely to be selected for that cell type relative to one or more other cell types (e.g., human cell types). Identified miRNA target sequences can be tested for their ability to suppress expression of a reporter such as GFP inserted into MVA under the control of an appropriate expression control sequence.

In some cases it will be desirable to use a plurality of different miRNA target sequences. The miRNA target sequences can be separated one from another by 1, 2, 3, 4, 5, 10 (or more) nucleotides. Expression in chicken embryo fibroblasts or other cells (e.g., other avian cells or avian stem cells) permissive for MVA production can be tested. In some embodiments expression is not suppressed in mammalian cells (e.g., 293T kidney cells, U937 monocytes) and for expression in human PBMC.

A database of miRNA sequences and access to miRNA target sequence information can be found on the internet at mirbase.org.

### MVA Vectors and HIV Polypeptides

The invention provides compositions (including pharmaceutically or physiologically acceptable compositions) that contain a MVA vector, having a polypeptide expression sequence. The polypeptide expression sequence can include one or more of the sequences described herein (the features of the polypeptide expression sequence and representative sequences are described at length below; any of these, or any combination of these, can be used as the polypeptide expression sequence). When the polypeptide expression sequence is expressed, the expressed polypeptide(s) may generate an immune response against one or more (e.g., two, three, four, five, or six) infectious agents, e.g., HIV

The invention also features compositions (including pharmaceutically or physiologically acceptable compositions) that contain, but are not limited to, two vectors: a first viral vector that encodes one or more (e.g., two, three, four, five, six, seven, eight, nine, or ten) polypeptides (i.e., a vector that includes a vaccine insert and/or a sequence expressing an immune modulator such as GM-CSF) that elicit (e.g., induces or enhances) an immune response against an HIV. A MVA vector can encode Gag-Pol or a modified form thereof. In addition, it can encode Gag-Pol and Env or modified forms thereof. The encoded HIV polypeptide can be a variant of a natural-occurring HIV polypeptide that includes one or more point mutations, insertions, or deletions. Particularly useful HIV polypeptide sequences include one or more (e.g., at least two, three, four, or five) safety mutations (e.g., deletion of the LTRs and of sequences encoding integrase (IN), Vif, Vpr, and Nef). The vectors can encode one or more (e.g., two, three, four, five, six, or seven) of Gag, PR, RT, Env, Tat, Rev, and Vpu proteins, one or more (e.g., two, three, four, five, six, or seven) of which may contain safety mutations (particular mutations are described at length below). Moreover, the isolated nucleic acids can be of any HIV Glade and nucleic acids from different clades can be used in combination (as described further below). In the work described herein, Glade B inserts are designated JS (e.g., JS2, JS7, and JS7.1), Glade AG inserts are designated IC (e.g., IC2, IC25, IC48, and IC90), and Glade C inserts are designated IN (e.g., IN2 and IN3). The viral vectors can also encode human GM-CSF (mwlqsllllg tvacsisapa rspspstqpw ehvnaiqear rllnlsrdta aemnetvevi semfdlqept clqtrlelyk qglrgsltkl kgpltmmash ykqhcpptpe tscatqiitf esfkenlkdf llvipfdcwe pvqe; SEQ ID NO: 10). A non-limiting example of a location for insertion of the GM-CSF is shown in FIG. 1.

Where the compositions contain MVA vectors that differ either in their backbone, regulatory elements, or insert(s), the ratio of the vectors in the compositions, and the routes by which they are administered, can vary. The ratio of one type of vector to another can be equal or roughly equal (e.g., roughly 1:1 or 1:1:1, etc.). Alternatively, the ratio can be in any desired proportion (e.g., 1:2, 1:3, 1:4 . . . 1:1100, 1:1000; 1:2:1, 1:3:1, 1:4:1 . . . 1:10:1, 1:100:1, 1:1000:1; etc.). Thus, the invention features compositions containing a variety of vectors, the relative amounts of antigen-expressing vectors being roughly equal or in a desired proportion. While preformed mixtures may be made (and may be more convenient), one can, of course, achieve the same objective by administering two or more (e.g., three, four, five, or six) vector-containing compositions (on, for example, the same occasion (e.g., within minutes of one another) or nearly the same occasion (e.g., on consecutive days)).

In any of the above described viral vectors, the polypeptide expression sequence can contain a sequence that encodes one or more (e.g., two, three, four, five, six, seven, eight, nine, or ten) polypeptide selected from the group of: gag, gp120, pol, env, Tat, Rev, Vpu, Nef, Vif, and Vpr. In additional embodiments of all the above vectors and polypeptide expression sequence, the one or more (e.g., two, three, four, five, six, seven, eight, nine, or ten) polypeptides (e.g., Gag, Env, Pol, Env, Tat, Rev, Vpu, Nef, Vif, and Vpr) is a mutant or a natural variant or a fragment of a natural polypeptide. In any of the above viral vectors and vaccine inserts, the polypeptide expression sequence can contain a sequence that encodes gag, pol, Tat, Rev, and env. In additional embodiments of the above vectors and vaccine inserts, the insert can contain a sequence that encodes gag, pol, tat, rev, env, and vpu.

In any of the above vectors or vaccine inserts, the encoded GM-CSF can be full-length human GM-CSF. In additional embodiments of the vectors and vaccine inserts, the sequence encoding GM-CSF can contain the sequence of: nucleotides 6633-7068 of SEQ ID NO: 7, nucleotides 6648-7082 of SEQ ID NO: 8, or nucleotides 7336-7770 of SEQ ID NO: 9. In any of the above viral vectors or polypeptide expression sequence, the encoded GM-CSF can be a truncated human GM-CSF or a mutant human GM-CSF that is capable of stimulating macrophage differentiation and proliferation, or activating polypeptide presenting cells.

The invention further provides methods of manufacturing a medicament for inducing an immune response in a subject using any of the above described vectors

By the term “natural variant” is meant a sequence that is naturally found in a subject or a virus. For example, human genes often contain single nucleotide polymorphisms that are present in certain individuals within a population. Viruses often acquire spontaneous mutations in their nucleic acid after serial passage in vitro or upon replication in an infected subject. Mutations within HIV sequences may confer resistance to drug treatment or alter the rate of infection or replication of the virus in a subject. Several natural variant sequences of HIV clades are known in the art (see, for example, the Los Alamos DNA Database website). By the term “mutant” is meant at least one (e.g., at least two, three, four, five, six, seven, eight, nine, ten, 100 or more) amino acid or nucleotide change in a sequence when compared to a wild type or predominant polypeptide or nucleotide sequence. A mutation may occur naturally in a cell or may be introduced by molecular biology techniques into a target sequence. The term mutant can include one or more (e.g., two, three, four, five, six, seven, eight, nine, or ten) amino acid or nucleotide deletions, additions, or substitutions.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. Schematic of a portion of a recombinant MVA virus. The MVA vaccine expresses gag-pol sequences in deletion III and env sequences in deletion II of MVA. Transcriptional control elements are shaded. For the MVA virus, transcription is under the control of the PmH5 early/late promoter. The MVA encodes an envelope glycoprotein that has been truncated for 146 amino acids at the C-terminus of its gp41 subunit. Xs, indicate inactivating point mutations in reverse transcriptase.

FIG. 2. Schematic of HIV polypeptides encoded by certain recombinant MVA virus. Point mutations and deletions in reverse transcriptase and packaging sequences in gag are indicated.

FIG. 3 is the nucleotide sequence encoding Env in MVA 65 A/G.

FIG. 4 is the nucleotide sequence encoding Gag/Pol in MVA 65 A/G.

FIG. 5 is the nucleotide sequence encoding Env in MVA 62 B.

FIG. 6 is the nucleotide sequence encoding Gag/Pol in MVA 62 B.

FIG. 7 is the nucleotide sequence encoding Env in MVA 71 C.

FIG. 8 is the nucleotide sequence encoding Gag/Pol in MVA 71 C.

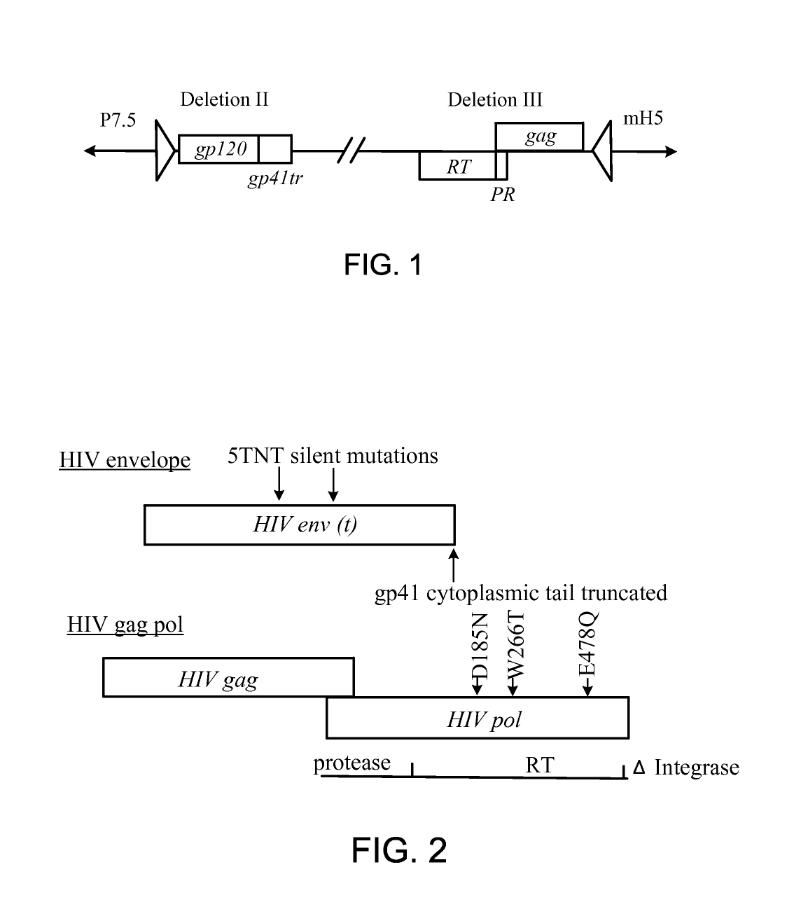

### DETAILED DESCRIPTION

This invention encompasses viral vectors each of which include one or more nucleic acid sequences that encode one or more polypeptides, e.g., polypeptides that elicit (e.g., that induce or enhance) an immune response against the pathogen from which the polypeptide was obtained or derived. Generally, the MVA will have polypeptide and/or GM-CSF encoding sequences inserted into Deletion II and Deletion III of MVA or between modified insertion sites or between essential genes or genes that affect virus replication to prevent overgrowth of MVAs that have lost inserts (Wyatt et al., J. Virol. 83: 7176-7184) described in more detail in US, application Ser. No. 12/377,847 filed 17 Feb. 2009. In order to suppress expression of polypeptides during manufacture, miRNA target sequences are preferably inserted into the 3′ untranslated region of each transcript.

The invention features the nucleic acid sequences disclosed herein, analogs thereof, and compositions containing those nucleic acids (whether vector plus polypeptide expression sequence or polypeptide expression sequence only; e.g., physiologically acceptable solutions, which may include carriers or other reagents used to deliver MVA to cells. The analogs can be sequences that are not identical to those disclosed herein, but that include the same or similar mutations (e.g., the same point mutation or a similar point mutation) at positions analogous to those included in the present sequences (e.g., any of the JS, IC, or IN sequences disclosed herein). A given residue or domain can be identified in various HIV clades even though it does not appear at precisely the same numerical position. The analogs can also be sequences that include mutations that, while distinct from those described herein, similarly inactivate an HIV gene product. For example, a gene that is truncated to a greater or lesser extent than one of the genes described here, but that is similarly inactivated (e.g., that loses a particular enzymatic activity) is within the scope of the present invention.

The pathogens and antigens, which are described in more detail in US-2003-0175292-A1 and US-2008-0193483-A1 (incorporated by reference), include human immunodeficiency viruses of any Glade (e.g. from any known Glade or from any isolate (e.g., Glade A, AG, B, C, D, E, F, G, H, I, J, K, or L)). Additional HIV sequences and mutant sequences are known in the art (e.g., the HIV Sequence Database in Los Alamos and the HIV RT/Protease Sequence Database in Stanford). Moreover, one or more of the inserts within any construct can be mutated to decrease their natural biological activity (and thereby increase their safety) in humans. At least one of the two or more sequences can be mutant or mutated so as to limit the encapsidation of viral RNA (preferably, the mutation(s) limit encapsidation appreciably). One can introduce mutations and determine their effect (on, for example, expression or immunogenicity) using techniques known in the art; polypeptides that remain well expressed (e.g., polypeptides that are expressed about as well as or better than their wild type counterparts), but are less biologically active than their wild type counterparts, are within the scope of the invention. Techniques are also available for assessing the immune response.

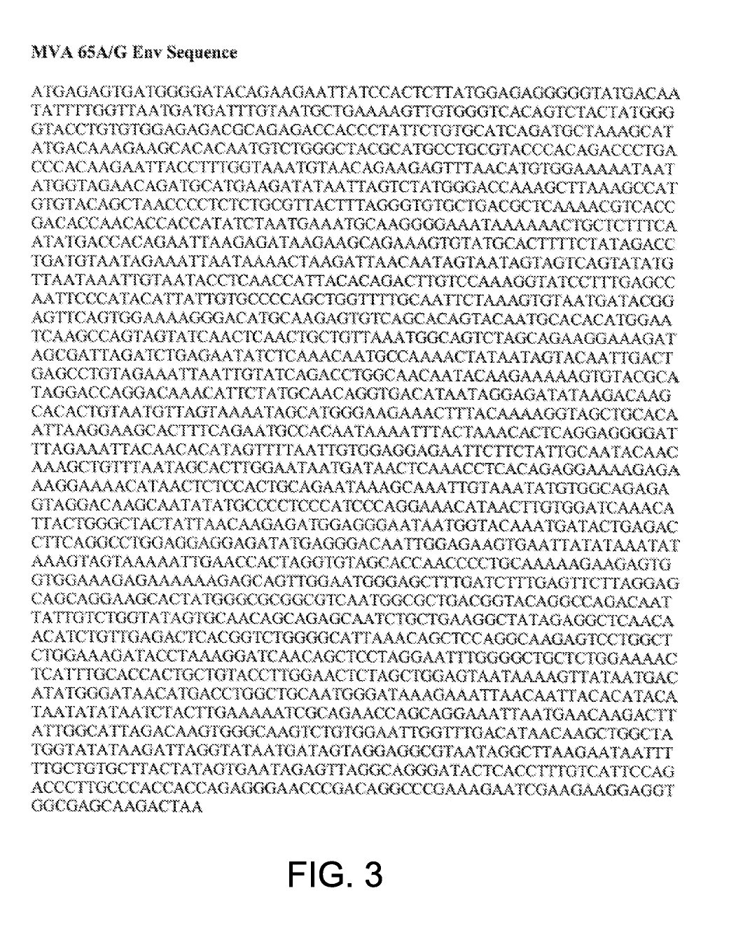

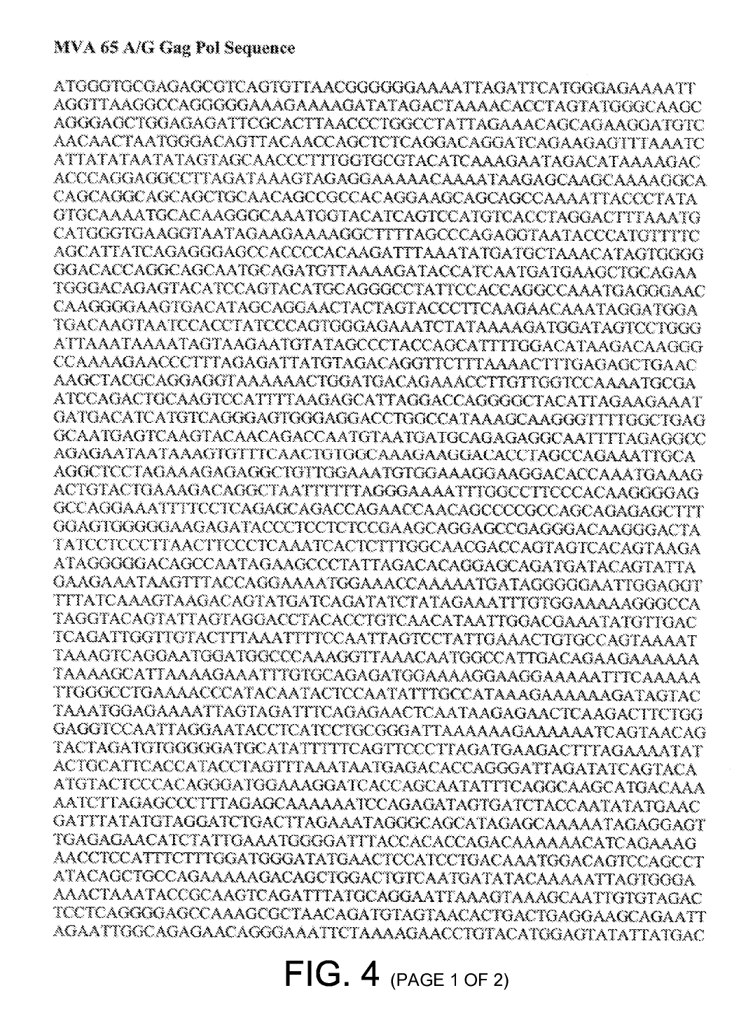

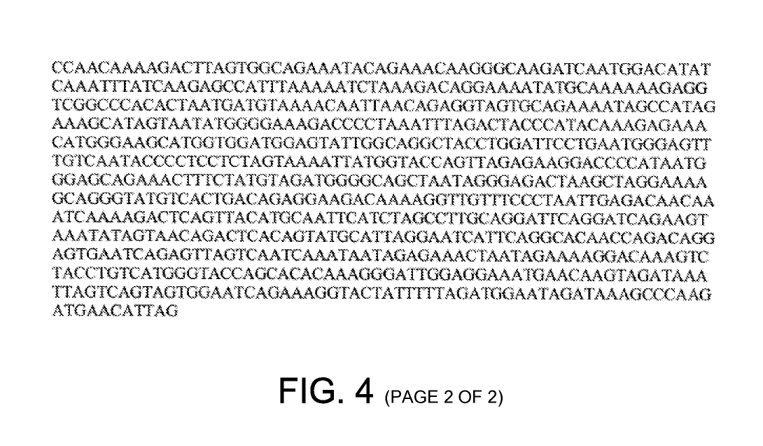

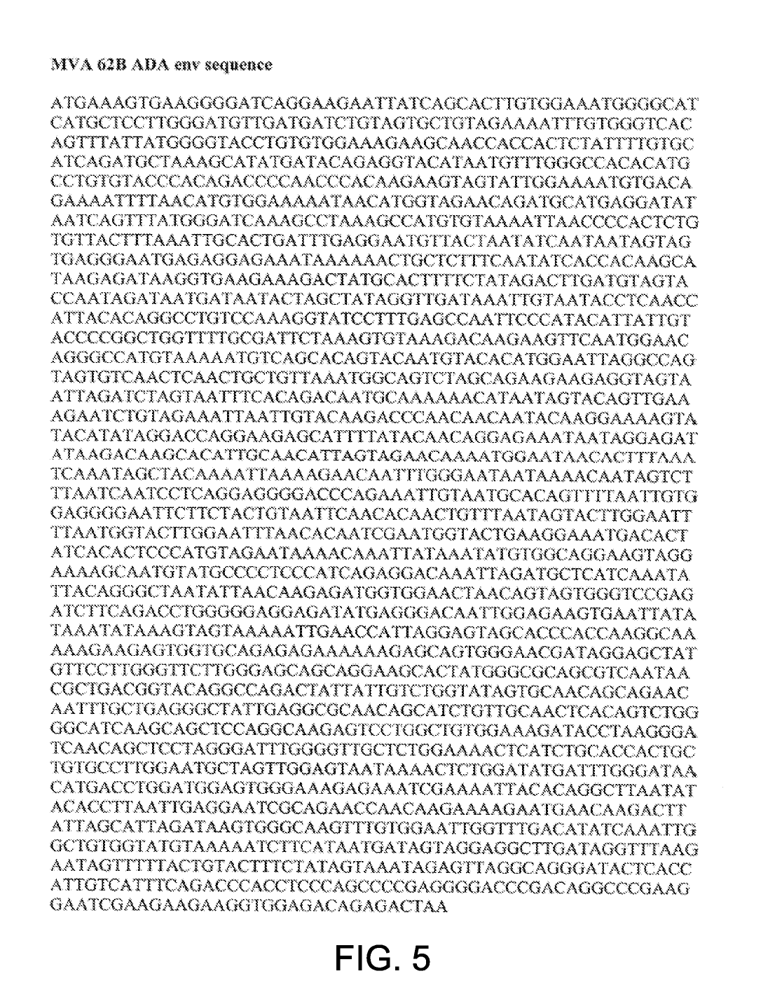

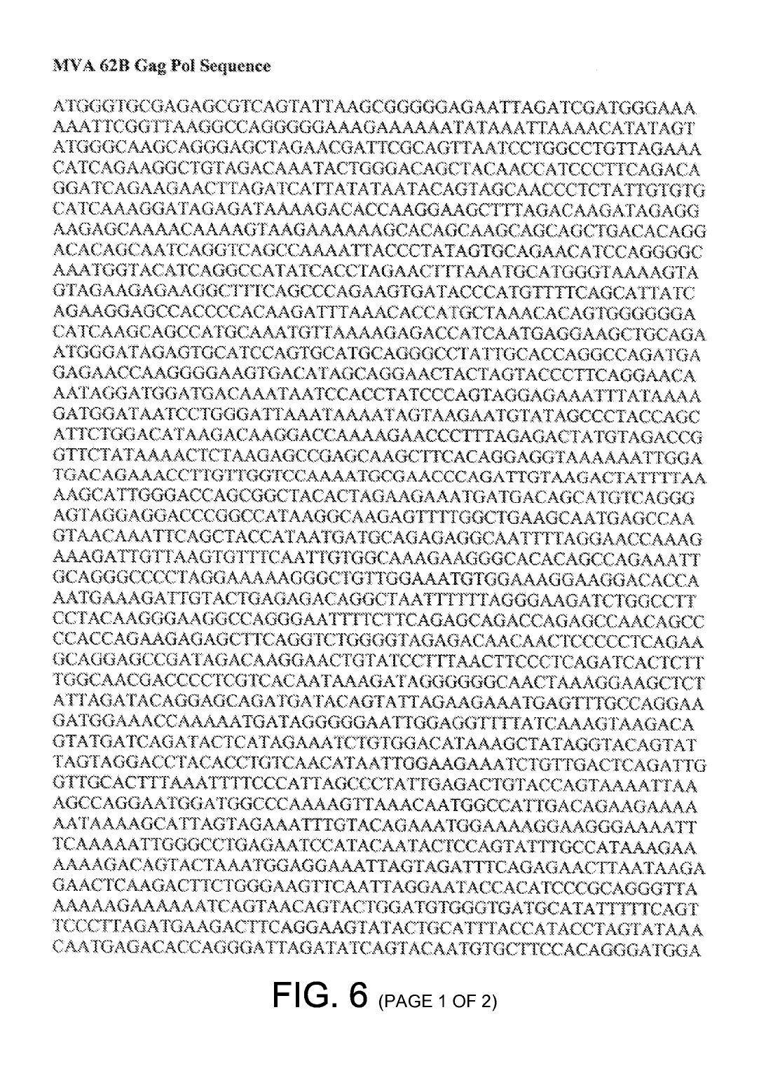

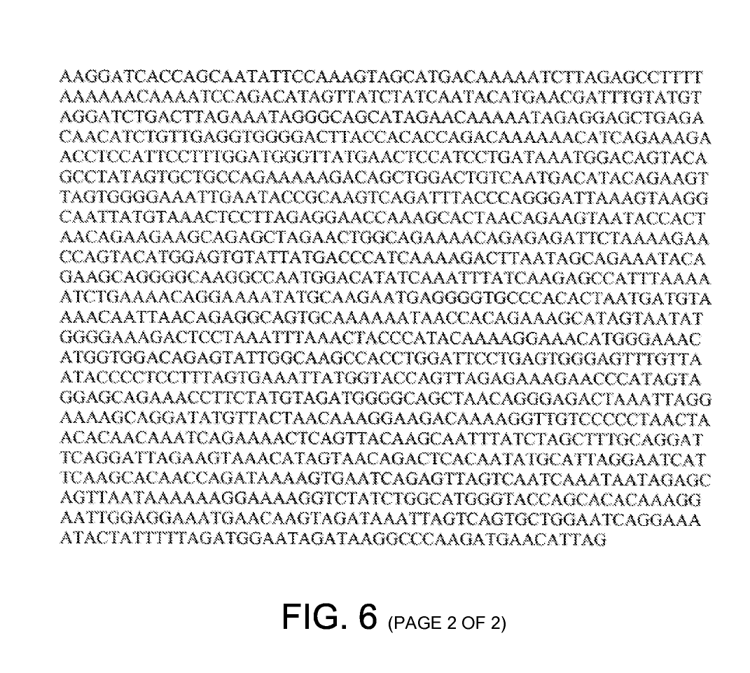

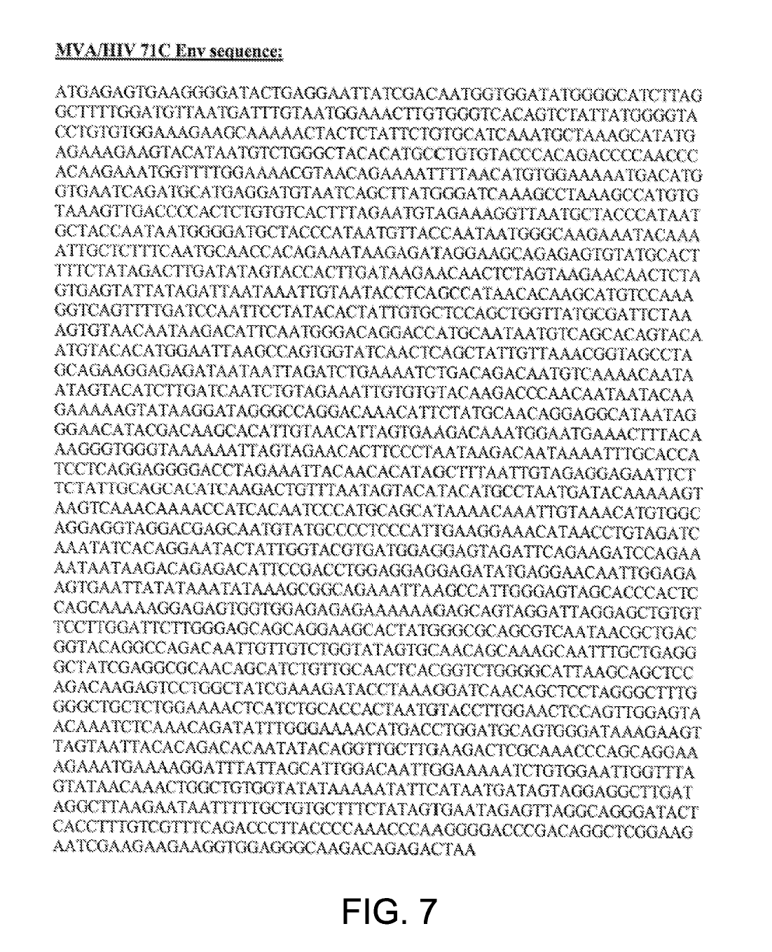

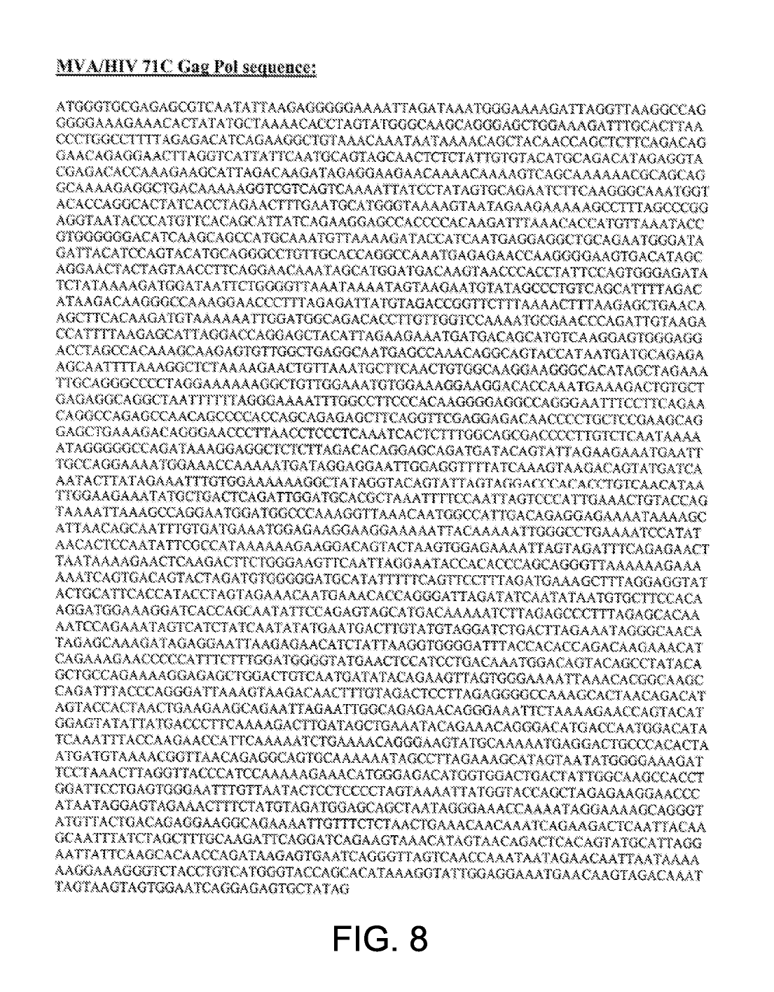

US-2008-0193483-A1 provides a detailed description of three different MVA vectors, MVA 65A/G, MVA 62B and MVA 71C, expressing HIV polypeptides. Each of these can be modified by insertion of miRNA target sequences. Each of the three vectors encodes Env and Gag/Pol with safety mutations. Sequences encoding these polypeptides are depicted in FIGS. 3-8.

The mutant constructs (e.g., a vaccine insert) can include sequences encoding one or more of the substitution mutants described herein (see, e.g. the Examples) or an analogous mutation in another HIV Glade. In addition to, or alternatively, HIV polypeptides can be rendered less active by deleting part of the gene sequences that encode them. Thus, the compositions of the invention can include constructs that encode polypeptides that, while capable of eliciting an immune response, are mutant (whether encoding a protein of a different length or content than a corresponding wild type sequence) and thereby less able to carry out their normal biological function when expressed in a patient. As noted above, expression, immunogenicity, and activity can be assessed using standard techniques in molecular biology and immunology.

The GM-CSF sequence included in the vectors and the vaccine inserts may be a full-length human GM-CSF (SEQ ID NO: 10) or may be a polypeptide that includes a sequence that is at least 95% identical to GM-CSF (SEQ ID NO: 10) and has one or more (e.g., two or three) biological activities of GM-CSF (e.g., capable of stimulating macrophage differentiation and proliferation, or activating polypeptide presenting cells). The GM-CSF may include one or more mutations (e.g., one or more (e.g., at least two, three, four, five, or six) amino acid substitutions, deletions, or additions)). Desirably, any mutant GM-CSF proteins also have one or more (e.g., two or three) biological activities of GM-CSF (as described above). Assays for the measurement of the biological activity of GM-CSF proteins are known in the art (see, e.g., U.S. Pat. No. 7,371,370; incorporated herein by reference in its entirety).

Particular polypeptides include the following. A polypeptide comprising a wild type or mutant gag sequence (e.g., a gag sequence having a mutation in one or more of the sequences encoding a zinc finger at one or more of the cysteine residues at positions 392, 395, 413, or 416 to another residue (e.g., serine) or the mutation can change one or more of the cysteine residues at positions 390, 393, 411, or 414 to another residue (e.g., serine). For HIV Pol it may be wild type or mutant Pol. The sequence can be mutated by deleting or replacing one or more nucleic acids, and those deletions or substitutions can result in a Pol gene product that has less enzymatic activity than its wild type counterpart (e.g., less integrase activity, less reverse transcriptase (RT) activity, or less protease activity). For example, one can inhibit RT by inactivating the polymerase's active site or by ablating strand transfer activity. Alternatively, or in addition, one can inhibit the polymerase's RNase H activity.

Where a polypeptide includes some or all of the pol sequence, another portion of the pol sequence that can optionally be altered is the sequence encoding the protease activity (regardless of whether or not sequences affecting other enzymatic activities of Pol have been altered). Where the composition includes either a viral vector with a polypeptide expression sequence or a polypeptide expression sequence alone, that polypeptide expression sequence can encode one or more of wild type or mutant Env, Tat, Rev, Nef, Vif, Vpr, or Vpu. With respect to Env, one or more mutations can be present. For example, one or more amino acids can be deleted from the gp120 surface and/or gp41 transmembrane cleavage products. With respect to Gag, one or more amino acids can be deleted from one or more of: the matrix protein (p17), the capsid protein (p24), the nucleocapsid protein (p7) and the C-terminal peptide (p6). For example, amino acids in one or more of these regions can be deleted. With respect to Pol, one or more amino acids can be deleted from the protease protein (p10), the reverse transcriptase protein (p66/p51), or the integrase protein (p32).

More specifically, the compositions of the invention can include a viral vector that encodes: (a) a Gag protein in which one or more of the zinc fingers has been inactivated to limit the packaging of viral RNA; (b) a Pol protein in which (i) the integrase activity has been inhibited by deletion of some or all of the pol sequence and (ii) the polymerase, strand transfer, and/or RNase H activity of reverse transcriptase has been inhibited by one or more point mutations within the pol sequence; and (c) Env, Tat, Rev, and Vpu, with or without mutations. In this embodiment, as in others, the encoded proteins can be obtained or derived from a subtype A, B or C HIV (e.g., HIV-1) or recombinant forms thereof. Where the compositions include non-identical vectors, the sequence in each type of vector can be from a different HIV Glade (or subtype or recombinant form thereof). For example, the invention features compositions that include plasmid vectors encoding the polypeptides just described (Gag-Pol, Env etc.), where some of the plasmids include polypeptides that are obtained from, or derived from, one Glade and other plasmids include polypeptides that are obtained (or derived) from another Glade. Mixtures representing two, three, four, five, six, or more clades (including all clades) are within the scope of the invention.

The encoded proteins can also be those of, or those derived from, any of HIV clades (or subtypes) E, F, G, H, I, J, K or L or recombinant forms thereof. An HIV-1 classification system has been published by Los Alamos National Laboratory (HIV Sequence Compendium-2001, Kuiken et al, published by Theoretical Biology and Biophysics Group T-10, Los Alamos, N. Mex., (2001)), more recent HIV sequences are available on the Los Alamos HIV sequence database website.

The compositions of the invention can also include a viral vector encoding: (a) a Gag protein in which one or both zinc fingers have been inactivated; (b) a Pol protein in which (i) the integrase activity has been inhibited by deletion of some or all of the pol sequence, (ii) the polymerase, strand transfer, and/or RNase H activity of reverse transcriptase has been inhibited by one or more point mutations within the pol sequence and (iii) the proteolytic activity of the protease has or has not been inhibited by one or more point mutations; and (c) Env, Tat, Rev, and Vpu, with or without mutations. As noted above, proteolytic activity can be inhibited by introducing a mutation at positions 1641-1643 of SEQ ID NO:8 or at an

Virus vaccine inserts of the present invention generate non-infectious VLPs (a term that can encompass true VLPs as well as aggregates of viral proteins) from a single DNA. This was achieved using the subgenomic splicing elements normally used by immunodeficiency viruses to express multiple gene products from a single viral RNA. The subgenomic splicing patterns are influenced by (i) splice sites and acceptors present in full length viral RNA, (ii) the Rev responsive element (RRE) and (iii) the Rev protein. The splice sites in retroviral RNAs use the canonical sequences for splice sites in eukaryotic RNAs. The RRE is an approximately 200 by RNA structure that interacts with the Rev protein to allow transport of viral RNAs from the nucleus to the cytoplasm. In the absence of Rev, the approximately 10 kb RNA of immunodeficiency virus mostly undergoes splicing to the mRNAs for the regulatory genes Tat, Rev, and Nef. These genes are encoded by exons present between RT and Env and at the 3′ end of the genome. In the presence of Rev, the singly spliced mRNA for Env and the unspliced mRNA for Gag and Pol are expressed in addition to the multiply spliced mRNAs for Tat, Rev, and Nef.

The expression of non-infectious VLPs from a single DNA affords a number of advantages to an immunodeficiency virus vaccine. The expression of a number of proteins from a single DNA affords the vaccinated host the opportunity to respond to the breadth of T- and B-cell epitopes encompassed in these proteins. The expression of proteins containing multiple epitopes allows epitope presentation by diverse histocompatibility types. By using whole proteins, one offers hosts of different histocompatibility types the opportunity to raise broad-based T cell responses. This may be essential for the effective containment of immunodeficiency virus infections, whose high mutation rate supports ready escape from immune responses (Evans et al., Nat. Med. 5:1270-1276, 1999; Poignard et al., Immunity 10:431-438, 1999, Evans et al., 1995). In the context of the present vaccination scheme, just as in drug therapy, multi-epitope T cell responses that require multiple mutations for escape will provide better protection than single epitope T cell responses (which require only a single mutation for escape).

Preferably, the viral vectors featured in the compositions and methods of the present invention are highly attenuated. Several attenuated strains of vaccinia virus were developed to avoid undesired side effects of smallpox vaccination. The modified vaccinia Ankara (MVA) virus was generated by long-term serial passages of the Ankara strain of vaccinia virus on chicken embryo fibroblasts (CVA; see Mayr et al., Infection 3:6-14, 1975). The MVA virus is publicly available from the American Type Culture Collection (ATCC; No. VR-1508; Manassas, Va.). The desirable properties of the MVA strain have been demonstrated in clinical trials (Mayr et al., Zentralbl. Bakteriol. 167:375-390, 1978; Stickl et al., Dtsch. Med. Wschr. 99:2386-2392, 1974; see also, Sutter and Moss, Proc. Natl. Acad. Sci. U.S.A. 89:10847-10851, 1992). During these studies in over 120,000 humans, including high-risk patients, no side effects were associated with the use of MVA vaccine.

The MVA vectors can be prepared as follows. A DNA construct that contains a DNA sequence that encodes a foreign polypeptide (e.g., any of the HIV polypeptides described herein) and that is flanked by MVA DNA sequences adjacent to a naturally occurring deletion within the MVA genome (e.g., deletion III or other non-essential site(s); six major deletions of genomic DNA (designated deletions I, II, III, IV, V, and VI) totaling 31,000 base pairs have been identified (Meyer et al., J. Gen. Virol. 72:1031-1038, 1991)) or flanked by MVA sequences adjacent to modified deletions sites or essential genes for virus replication (see end for patent references) is introduced into cells infected with MVA under conditions that permit homologous recombination to occur. Once the DNA construct has been introduced into the eukaryotic cell and the foreign DNA has recombined with the viral DNA, the recombinant vaccinia virus can be isolated by methods known in the art (isolation can be facilitated by use of a detectable marker). The DNA constructed to be inserted can be linear or circular (e.g., a plasmid, linearized plasmid, gene, gene fragment, or modified HIV genome). The foreign DNA sequence is inserted between the sequences flanking the naturally occurring deletion, modifications of the naturally occurring deletions or essential genes for virus growth. For better expression of a DNA sequence, the sequence can include regulatory sequences (e.g., a promoter, such as the promoter of the vaccinia 11 kDa gene or the 7.5 kDa gene or a modified promoter such as mH5). The DNA construct can be introduced into MVA-infected cells by a variety of methods, including calcium phosphate-assisted transfection (Graham et al., Virol. 52:456-467, 1973 and Wigler et al., Cell 16:777-785, 1979), electroporation (Neumann et al., EMBO J. 1:841-845, 1982), microinjection (Graessmann et al., Meth. Enzymol. 101:482-492, 1983), by means of liposomes (Straubinger et al., Meth. Enzymol. 101:512-527, 1983), by means of spheroplasts (Schaffner, Proc. Natl. Acad. Sci. U.S.A. 77:2163-2167, 1980), or by other methods known in the art.

What is claimed is:

1. A recombinant MVA virus comprising a first polypeptide expression sequence comprising: a promoter, a sequence encoding at least one polypeptide, and one or more miRNA target sequences, wherein the one or more miRNA target sequences are located in a 3′ transcribed, non-translated portion of the first polypeptide expression sequence.

2. The recombinant MVA virus of claim 1 comprising a second polypeptide expression sequence comprising: a promoter, a sequence encoding at least one polypeptide and one or more miRNa target sequences, wherein the one or more miRNA target sequences are located in a 3′ transcribed, non-translated portion of the second polypeptide expression sequence.

3. The recombinant MVA virus of claim 2 wherein the first polypeptide expression sequence and the second polypeptide expression sequence are inserted into a naturally-occurring deletion of MVA selected from Deletion I, II, III or modified III, IV, V and VI or between two essential genes for virus replication such as 18G1, provided that the first and the second polypeptide expression sequences are not inserted into the same site of MVA.

4. The recombinant MVA virus of claim 1 wherein the one or more miRNA sequences reduce expression of the polypeptide when the recombinant MVA is grown in an avian cell.

5. The recombinant MVA virus of claim 4 wherein the one or more miRNA sequences reduce expression of the at least one polypeptide when the recombinant MVA is grown in an cultured avian cell by at least 20% compared to expression of the at least one polypeptide by an otherwise identical recombinant MVA lacking the one or more miRNA target sequences.

6. The recombinant MVA virus of claim 4 where the one or more miRNA target sequences reduce expression of the at least one polypeptide by when the recombinant MVA is grown in a cultured human cell by less than 10% compared to expression of the at least one polypeptide by an otherwise identical recombinant MVA lacking the one or more miRNA target sequences.

7. The recombinant MVA virus of claim 1 wherein the one or more miRNA target sequences comprise at least two different miRNA target sequences.

8. The recombinant MVA virus of claim 1 wherein the polypeptide expression sequence comprises a sequence encoding a polypeptide selected from the group consisting of: HIV Gag, HIV gp120, HIV Pol, HIV env, HIV Tat, HIV Rev, HIV Vpu, HIV Nef, HIV Vif, HIV Vpr, and fragments thereof comprising at least 20 amino acids.

9. The recombinant MVA virus of claim 2 wherein the first and the second polypeptide expression sequence comprises a sequence encoding a polypeptide selected from the group consisting of: HIV Gag, HIV gp120, HIV Pol, HIV env, HIV Tat, HIV Rev, HIV Vpu, HIV Nef, HIV Vif, HIV Vpr, and fragments thereof comprising at least 20 amino acids.

10. The recombinant MVA virus of claim 2 wherein the first or the second polypeptide expression sequence comprises a sequence encoding a human immune modulator.

11. The recombinant MVA virus of claim 2 wherein the first polypeptide expression sequence comprises a sequence encoding HIV Env and the second polypeptide expression sequence comprises a sequence encoding HIV Gag and HIV Pol.

12. The recombinant MVA virus of claim 11 wherein the first polypeptide expression sequence is inserted into Deletion II and the second polypeptide expression sequence is inserted into Deletion III.

13. The recombinant MVA virus of claim 1 wherein the promoter is a viral promoter such as a poxvirus promoter.

14. A method for producing MVA comprising providing a cultured avian cell infected with the recombinant MVA virus of any of claims 1-13; culturing the cell under conditions that are permissive for replication of MVA virus; and isolating the MVA virus from the cultured cell.

15. A method for producing a pharmaceutical composition comprising MVA, the methods comprising providing a cultured avian cell infected with the recombinant MVA virus of any of claims 1-13; culturing the cell under conditions that are permissive for replication of MVA virus; isolating the MVA virus from the cultured cell; and combining the isolated MVA virus with a pharmaceutically acceptable carrier or diluent.
